(19) United States
(12) Patent Application Publication
(10) Pub. No.: US 2016/0015609 A1
(43) Pub. Date: Jan. 21, 2016

(54) NOVEL WATER-IN-OIL EMULSIONS WITH A HIGH CONTENT OF AQUEOUS PHASE AND OF LIQUID CONSISTENCY, WHICH ARE STABLE ON STORAGE

(71) Applicant: SOCIETE D'EXPLOITATION DE PRODUITS POUR LES INDUSTRIES CHIMIQUES SEPPIC, Paris Cedex 07 (FR)

(72) Inventors: Emmanuelle MERAT, LAUTREC (FR); Severine SIGURANI, CASTRES (FR)

(21) Appl. No.: 14/782,073

(22) PCT Filed: Mar. 21, 2014
(86) PCT No.: PCT/FR2014/050668

§ 371 Date: Oct. 2, 2015

(30) Foreign Application Priority Data
Apr. 12, 2013 (FR) 1353352

(51) Int. Cl. A61K 8/06; A61Q 19/00; A61K 8/60; A61Q 13/00; A61Q 19/02; A61K 8/81; A61Q 17/04; A61Q 15/00

(52) U.S. Cl. CPC A61K 8/064; A61Q 17/04; A61Q 19/00; A61Q 15/00; A61Q 13/00; A61Q 19/02; A61K 8/8158; A61K 8/604; A61K 2800/596

(57) ABSTRACT

An oil-in-water emulsion including, by 100% of its mass: 60% to 98% of aqueous phase (A₁), including 0.05% to 1.65% of a cross-linked anionic polyelectrolyte (P) resulting from the polymerisation, in the presence of at least one cross-linking agent, of partially or completely salified 2-methyl-2-[(1-oxo-2-propenyl)-amino]-1-propanesulphonic acid with at least one neutral monomer selected from N,N-dialkyl acrylamides, wherein each alkyl group has between one and four carbon atoms, and at least one monomer of formula (I): R=radical C8-C20 and 1≦n≦20; 2% to 40% of fatty phase (A₂), including 1.25% to 50% of an emulsifying system (S) with at least one surfactant selected from alkylpolyglycoside compositions, alkylpolyglycoside and fatty alcohol compositions, polyglycerol esters, alkoxylated polyglycerol esters, polyglycolpolyhydroxystearates, polyglycerol polyhydroxystearates, alkoxylated polyglycerol polyhydroxystearates, polyethylene glycol/alkyl glycol copolymers; 50% to 98.75% of at least one oil and, optionally, at least one wax. Cosmetics including the emulsion.

The present invention relates to novel compositions in the form of water-in-oil emulsions, and also to the uses thereof in cosmetics, in pharmaceuticals, in the textile industry and in the paper industry.

Cosmetic, dermocosmetic, dermopharmaceutical and pharmaceutical compositions may be in the form of aqueous solutions, emulsions or powders. Emulsions are particularly preferred forms since they make it possible to convey both the water-soluble substances and the liposoluble substances that are commonly used in these applications. Oil-in-water (O/W) emulsions in which the continuous phase consists of a hydrophilic phase, generally an aqueous phase, and the phase dispersed with a lipophilic fatty phase, and water-in-oil (W/O) emulsions in which the continuous phase consists of a lipophilic fatty phase and the phase dispersed with a hydrophilic phase, generally an aqueous phase, are distinguished.

Oil-in-water emulsions are intrinsically more stable than water-in-oil emulsions; water-in-oil emulsions nevertheless have many advantages. Specifically, separation between water droplets reduces the possibility of proliferation of microorganisms. Furthermore, the use of preserving agents, which is essential when the continuous phase is aqueous, may be avoided, or lessened, when the continuous phase is fatty. Water-in-oil emulsions are much less sensitive to a low temperature than oil-in-water emulsions. Finally, an oily continuous phase makes it possible to cover the skin after application of the water-in-oil emulsion, which protects it from dehydration and against external substances, by forming a persistent oily film.

The patent published under the number U.S. Pat. No. 5,746,945 discloses water-in-oil emulsions that are stabilized with a two-component emulsifying system, a polysiloxane polyalkyl polyether copolymer and a phthalic anhydride derivative (a monoamide). They are prepared by gradual addition of the aqueous phase to the oily phase, these two phases having been beforehand at 71-74° C. (160 to 165° F.).

The international patent application published under the number WO 97/40814 discloses water-in-oil emulsions intended especially to be used for impregnating baby wipes and in which the emulsifiers used are of carboxylic acid type, substituted with hydrocarbons, or ABA “block” copolymers, involving monomers such as 12-hydroxystearic acid and ethylene glycol, or an alkyldimethicone copolyol.

The European patent application published under the number EP 1 459 801 A2 discloses the preparation of water-in-oil emulsions comprising, per 100% of their mass, from 60% to 98% by mass of aqueous phase gelled by means of the presence therein of a crosslinked polyelectrolyte.

However, the emulsion obtained is not sufficiently fluid and it does not give on the skin an oily film that is sufficiently persistent.

This is why the inventors have sought to develop novel W/O emulsions that do not have the drawbacks mentioned above, which remain homogeneous at room temperature after storage for a minimum of three months; whose dynamic viscosity, measured using a Brookfield LV™ viscometer equipped with a suitable spindle and at a speed of 6 rpm, is at 20° C. between 500 mPa·s and 40 000 mPa·s; that this dynamic viscosity is not 75% less than the dynamic viscosity measured under the same conditions after only 7 days of storage; whose consistency criterion is evaluated as “liquid” before its application to the skin, the evaluation being made according to the protocol described in paragraph II-2.3.1 of the experimental section of the present description; and/or whose flow threshold value, measured under the experimental conditions as described in paragraph II-2.3.2 of the experimental section of the present description, is strictly less than 40 Pa. This is why, according to a first aspect, a subject of the invention is a composition (E₁) which is in the form of a water-in-oil emulsion, characterized in that it comprises per 100% of its mass:
from 60% to 98% by mass of an aqueous phase (A₁) comprising, per 100% of its mass, from 0.05% to 1.65% by mass of a crosslinked anionic polyelectrolyte (P) derived from the polymerization, in the presence of at least one crosslinking agent, of 2-methyl-2-[(1-oxo-2-propenyl)amino]-1-propanesulfonic acid which is partially or totally salified, with at least one neutral monomer chosen from N,N-dialkylacrylamides, in which each of the alkyl groups comprises between one and four carbon atoms, and at least one monomer of formula (I):

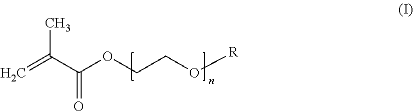

in which R represents a linear or branched alkyl radical comprising from eight to twenty carbon atoms and n represents an integer greater than or equal to one and less than or equal to twenty;
from 2% to 40% by mass of a fatty phase (A₂) comprising, per 100% of its mass, from 1.25% to 50% by mass of an emulsifying system (S) comprising one or more emulsifying surfactants selected from alkylpolyglycoside compositions, alkylpolyglycoside and fatty alcohol compositions, polyglycerol esters, alkoxylated polyglycerol esters, polyglycol polyhydroxystearates, polyglycerol polyhydroxystearates, alkoxylated polyglycerol polyhydroxystearates, polyethylene glycol-alkyl glycol copolymers; from 50% to 98.75% by mass of at least one oil and optionally of at least one wax.

The term “oil” denotes, in the definition of composition (E₁) that is the subject of the present invention, a compound or a mixture of compounds that is insoluble in water, and liquid at 25° C., and more particularly:
mineral oils such as liquid paraffin, liquid petroleum jelly, isoparaffins or white mineral oils;
oils of animal origin, such as squalene or squalane;
plant oils such as phytosqualane, sweet almond oil, copra oil, castor oil, jojoba oil, olive oil, rapeseed oil, groundnut oil, sunflower oil, wheat germ oil, corn germ oil, soybean oil, cotton seed oil, alfalfa oil, poppy oil, pumpkin oil, evening primrose oil, millet oil, barley oil, rye oil, safflower oil, candlenut oil, passion flower oil, hazelnut oil, palm oil, shea butter, apricot kernel oil, beauty-leaf oil, sisymbrium oil, avocado oil, calendula oil, and oils derived from flowers or vegetables;
ethoxylated plant oils;
synthetic oils, for instance fatty acid esters such as butyl myristate, propyl myristate, isopropyl myristate, cetyl myristate, isopropyl palmitate, octyl palmitate, butyl stearate, hexadecyl stearate, isopropyl stearate, octyl stearate, isocetyl stearate, dodecyl oleate, hexyl laurate or propylene glycol dicaprylate, lanolic acid-based esters, such as isopropyl lanolate, isocetyl lanolate, fatty acid monoglycerides, diglycerides and triglycerides, for instance glyceryl triheptanoate, alkylbenzoates, hydrogenated oils, poly(α-olefin), polyolefins such as poly(isobutane), synthetic isoalkanes such as isohexadecane and isododecane, and perflouro oils, and
silicone oils, for instance dimethylpolysiloxanes, methylphenylpolysiloxanes, silicones modified with amines, silicones modified with fatty acids, silicones modified with alcohols, silicones modified with alcohols and fatty acids, silicones modified with polyether groups, epoxy-modified silicones, silicones modified with fluoro groups, cyclic silicones and silicones modified with alkyl groups.

When composition (E₁) as defined above comprises a wax, this wax is particularly chosen from beeswax, carnauba wax, candelilla wax, ouricury wax, Japan wax, cork fiber wax, sugarcane wax, paraffin waxes, lignite waxes, microcrystalline waxes or lanolin wax; ozocerite; polyethylene wax; silicone waxes; plant waxes; fatty alcohols and fatty acids that are solid at room temperature; glycerides that are solid at room temperature.

The term “crosslinked anionic polyelectrolyte (P)” denotes, in the definition of composition (E₁) that is the subject of the present invention, a nonlinear crosslinked anionic polyelectrolyte, in the form of a three-dimensional network that is insoluble in water, but swellable in water and which leads to the production of a chemical gel.

The term “partially salified or totally salified” means, in the definition of the crosslinked anionic polyelectrolyte (P) present in composition (E₁) as defined above, that said 2-methyl-2-[(1-oxo-2-propenyl)amino]-1-propanesulfonic acid is partially or totally salified, especially in alkali metal salt form, for example in sodium salt or potassium salt form, or in ammonium salt form.

The term “neutral monomer chosen from N,N-dialkylacrylamides in which each of the alkyl groups comprises between one and four carbon atoms” denotes, in the definition of composition (E₁) that is the subject of the present invention, more particularly N,N-dimethylacrylamide, N,N-diethylacrylamide, N,N-dipropylacrylamide or N,N-diisopropylacrylamide.

According to a particular aspect of the present invention, composition (E₁) as defined previously is characterized in that it comprises, per 100% of its mass:
from 60% to 98% by mass of an aqueous phase (A₁) comprising, per 100% of its mass, from 0.05% to 1.65% by mass of a crosslinked anionic polyelectrolyte (P) derived from the polymerization, in the presence of a crosslinking agent, of 2-methyl-2-[(1-oxo-2-propenyl)amino]-1-propanesulfonic acid, which is partially or totally salified, with a neutral monomer chosen from N,N-dialkylacrylamides, in which each of the alkyl groups comprises between one and four carbon atoms, and a monomer of formula (I):

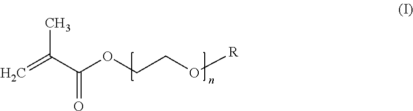

in which R represents a linear or branched alkyl radical comprising from eight to twenty carbon atoms and n represents an integer greater than or equal to one and less than or equal to twenty;
from 2% to 40% by mass of a fatty phase (A₂) comprising, per 100% of its mass, from 1.25% to 50% by mass of an emulsifying system (S) comprising one or more emulsifying surfactants selected from alkylpolyglycoside compositions, alkylpolyglycoside and fatty alcohol compositions, polyglycerol esters, alkoxylated polyglycerol esters, polyglycol polyhydroxystearates, polyglycerol polyhydroxystearates, alkoxylated polyglycerol polyhydroxystearates, polyethylene glycol-alkyl glycol copolymers; from 50% to 98.75% by mass of an oil and optionally of a wax.

According to a particular aspect of the present invention, in composition (E₁) as defined above, said crosslinked anionic polyelectrolyte (P) comprises, per 100 mol % of its constituent units, from 5 mol % to 95 mol % of monomer units derived from 2-methyl-2-[(1-oxo-2-propenyl)amino]-1-propanesulfonic acid, which is partially or totally salified, more particularly from 10 mol % to 90 mol % and most particularly from 20 mol % to 80 mol %.

According to another particular aspect of the present invention, in composition (E₁) as defined above, said crosslinked anionic polyelectrolyte (P) comprises, per 100 mol % of its constituent monomers, from 4.9 mol % to 90 mol % of monomer units derived from at least one neutral monomer chosen from N,N-dialkylacrylamides, in which each of the alkyl groups comprises between one and four carbon atoms, more particularly from 9.5 mol % to 85 mol % and most particularly from 15 mol % to 75 mol %.

According to another particular aspect of the present invention, in composition (E₁) that is the subject of the present invention, said crosslinked anionic polyelectrolyte (P) as defined previously comprises, per 100 mol % of its constituent monomers, from 0.1 mol % to 10 mol % and more particularly from 0.5 mol % to 5 mol % of monomer units derived from the monomer of formula (I).

A subject of the invention is more particularly a composition (E₁) as defined previously, characterized in that said crosslinked anionic polyelectrolyte (P) comprises, per 100 mol % of its constituent monomers:
from 20 mol % to 80 mol % of monomer units derived from 2-methyl-2-[(1-oxo-2-propenyl)amino]-1-propanesulfonic acid, which is partially or totally salified;
from 15 mol % to 75 mol % of monomer units derived from at least one neutral monomer chosen from N,N-dialkylacrylamides, in which each of the alkyl groups comprises between one and four carbon atoms;
from 0.5 mol % to 5 mol % of monomer units derived from at least one monomer of formula (I).

In formula (I) of the monomer present in said crosslinked anionic polyelectrolyte (P) included in composition (E₁) that is the subject of the present invention, the term “linear or branched alkyl radical comprising from eight to twenty carbon atoms” more particularly denotes for R:
either a radical derived from linear primary alcohols, for instance the octyl, decyl, undecyl, dodecyl, tridecyl, tetradecyl, pentadecyl, hexadecyl, heptadecyl, octadecyl, nonadecyl or eicosyl radical;
or a radical derived from Guerbet alcohols, which are branched 1-alkanols corresponding to the general formula:

CH₃—(CH₂)ₚ—CH[CH₃—(CH₂)ₚ₋₂]—CH₂OH,

in which p represents an integer between 2 and 9, for instance 2-ethylhexyl, 2-propylheptyl, 2-butyloctyl, 2-pentylnonyl, 2-hexyldecyl or 2-octyldodecyl radicals;
or a radical derived from isoalkanols corresponding to the general formula:

CH₃—CH(CH₃)—(CH₂)ₘ—CH₂OH,

in which m is an integer between 2 and 16, for instance 4-methylpentyl, 5-methylhexyl, 6-methylheptyl, 15-methylpentadecyl or 16-methylheptadecyl radicals, or 2-hexyloctyl, 2-octyldecyl or 2-hexyldodecyl radicals.

According to another particular aspect, composition (E₁) as defined previously is characterized in that said neutral monomer is N,N-dimethylacrylamide.

According to another particular aspect, a subject of the invention is a composition (E₁) as defined previously, characterized in that, in said crosslinked anionic polyelectrolyte (P) and for said monomer of formula (I) as defined previously, R represents an alkyl radical comprising from 12 to 18 carbon atoms.

According to an even more particular aspect, a subject of the invention is a composition (E₁) as defined previously, characterized in that in said crosslinked anionic polyelectrolyte (P) and for said monomer of formula (I) as defined previously, R represents an alkyl radical chosen from the elements of the group consisting of the dodecyl, tridecyl, tetradecyl, hexadecyl and octadecyl radicals.

According to another particular aspect, a subject of the invention is a composition (E₁) as defined previously, characterized in that in said crosslinked anionic polyelectrolyte (P) and for said monomer of formula (I) as defined previously, n represents an integer greater than or equal to 3 and less than or equal to 20.

According to an even more particular aspect, composition (E₁) as defined previously is characterized in that said monomer of formula (I) is tetraethoxylated lauryl methacrylate.

According to another even more particular aspect, a subject of the invention is a composition (E₁) as defined previously, characterized in that in said crosslinked anionic polyelectrolyte (P) as defined previously, said monomer of formula (I) is eicosaethoxylated stearyl methacrylate.

According to another particular aspect, a subject of the invention is a composition (E₁) as defined previously, characterized in that in said crosslinked anionic polyelectrolyte (P) as defined previously is crosslinked with a diethylenic or polyethylenic compound in the molar proportion expressed relative to the monomers used, is from 0.005 mol % to 1 mol %, more particularly from 0.01 mol % to 0.5 mol % and most particularly from 0.01 mol % to 0.25 mol %. The crosslinking agent is more particularly chosen from ethylene glycol dimethacrylate, tetraallyloxyethane, ethylene glycol diacrylate, diallylurea, triallylamine, trimethylolpropane triacrylate and methylenebis(acrylamide), or a mixture of these compounds.

The crosslinked anionic polyelectrolyte (P) used in composition (E₁) that is the subject of the present invention may also comprise various additives, such as complexing agents, transfer agents or chain-limiting agents. The transfer agents or chain-limiting agents are more particularly chosen from the group consisting of sodium hypophosphite, low molecular weight alcohols, for example methanol, ethanol, 1-propanol, isopropanol, butanol, thiols, for example 2-mercaptoethanol, transfer agents comprising a sulfate function, for example sodium methallylsulfonate, or mixtures of said transfer agents. The transfer agents or chain-limiting agents are more particularly used in molar proportions, expressed relative to the total number of moles of monomers used, of from 0.001 mol % to 1 mol %, more particularly from 0.001 mol % to 0.5 mol % and most particularly from 0.001 mol % to 0.1 mol %.

According to a particular aspect, a subject of the invention is a composition (E₁) as defined previously, characterized in that said crosslinked anionic polyelectrolyte (P) as defined previously is chosen from terpolymers of 2-methyl-2-[(1-oxo-2-propenyl)amino]-1-propanesulfonic acid partially salified in ammonium salt form, of N,N-dimethylacrylamide and of tetraethoxylated lauryl methacrylate, crosslinked with trimethylolpropane triacrylate or terpolymers of 2-methyl-2-[(1-oxo-2-propenyl)amino]-1-propanesulfonic acid partially salified in ammonium salt form, of N,N-dimethylacrylamide and of eicosaethoxylated stearyl methacrylate, crosslinked with trimethylolpropane triacrylate.

According to a more particular aspect, a subject of the invention is a composition (E₁) as defined previously, characterized in that said crosslinked anionic polyelectrolyte (P) is a terpolymer of 2-methyl-2-[(1-oxo-2-propenyl)amino]-1-propanesulfonic acid partially salified in ammonium salt form, of N,N-dimethylacrylamide and of tetraethoxylated lauryl methacrylate, crosslinked with trimethylolpropane triacrylate.

According to another more particular aspect, a subject of the invention is a composition (E₁) as defined previously, characterized in that said crosslinked anionic polyelectrolyte (P) comprises, per 100 mol %:
from 60 mol % to 80 mol % of monomer units derived from 2-methyl-2-[(1-oxo-2-propenyl)amino]-1-propanesulfonic acid partially salified in ammonium form,
from 15 mol % to 39.5 mol % of monomer units derived from N,N-dimethylacrylamide, and
from 0.5 mol % to 5 mol % of monomer units derived from tetraethoxylated lauryl methacrylate.

In the definition of composition (E₁) that is the subject of the present invention, the term “alkyl polyglycoside compositions” denotes a composition (C₁) represented by formula (II):

R₁—O-(G)ₓ-H (II)

in which x represents a decimal number between 1.05 and 5, G represents a reducing sugar residue and R₁ represents a saturated or unsaturated, linear or branched hydrocarbon-based aliphatic radical, optionally substituted with one or more hydroxyl groups comprising from 12 to 36 carbon atoms, said composition (C₁) consisting of a mixture of compounds represented by formulae (I₁), (I₂), (I₃), (I₄) and (I₅):

R₁—O-(G)₁-H (II₁)

R₁—O-(G)₂-H (II₂)

R₁—O-(G)₃-H (II₃)

R₁—O-(G)₄-H (II₄)

R₁—O-(G)₅-H (II₅)

in the respective molar proportions a₁, a₂, a₃, a₄ and a₅ such that:
the sum a₁+a₂+a₃+a₄+a₅ is equal to 1 and that
the sum a₁+2a₂+3a₃+4a₄+5a₅ is equal to x.

The term “saturated or unsaturated, linear or branched hydrocarbon-based aliphatic radical comprising from 12 to 36 carbon atoms, optionally substituted with one or more hydroxyl groups” denotes for the radical R₁ in formula (II) as defined above:
saturated linear alkyl radicals, for example n-dodecyl, n-tridecyl, n-tetradecyl, n-pentadecyl, n-hexadecyl, n-heptadecyl, n-octadecyl, n-nonadecyl, n-eicosyl or n-docosyl radicals;
unsaturated linear radicals such as dodecenyl, tridecenyl, tetradecenyl, pentadecenyl, hexadecenyl, heptadecenyl, octadecenyl, nonadecenyl, eicosenyl, docosenyl, 4-dodecenyl or 5-dodecenyl radicals;
saturated or unsaturated, linear or branched aliphatic radicals comprising from 12 to 36 carbon atoms substituted with one or two hydroxyl groups, such as hydroxydodecyl, hydroxytetradecyl, hydroxyhexadecyl, hydroxyoctadecyl, hydroxyeicosyl or hydroxydocosyl radicals, for example the 12-hydroxyoctadecyl radical;
radicals derived from the isoalkanols of formula (1):

(CH₃)(CH₃)CH—(CH₂)ᵣ—CH₂—OH (1)

in which r represents an integer between 8 and 20, for example isodecyl, isoundecyl, isododecyl, isotridecyl, isotetradecyl, isopentadecyl, isohexadecyl, isopentadecyl, isooctadecyl, isononadecyl, isoeicosyl or isodocosyl radicals;
branched alkyl radicals derived from Guerbet alcohols, of formula (2):

CH(CₛH₂ₛ₊₁)(CₜH₂ₜ₊₁)—CH₂—OH (2)

in which t is an integer between 6 and 18, s is an integer between 4 and 18 and the sum s+t is greater than or equal to 10 and less than or equal to 22, for example 2-butyloctyl, 2-butyldecyl, 2-hexyloctyl, 2-hexyldecyl, 2-octyldecyl, 2-hexyldodecyl, 2-octyldodecyl, 2-decyltetradecyl, 2-dodecylhexadecyl or 2-tetradecyloctadecyl radicals.

According to a particular aspect, in the definition of formula (II) as defined above, R₁ represents a saturated or unsaturated, linear or branched hydrocarbon-based aliphatic radical comprising from 12 to 24 carbon atoms.

The term “reducing sugar” in the definition of formula (II) as defined above denotes saccharide derivatives that do not have in their structures any glycoside bond established between an anomeric carbon and the oxygen of an acetal group as are defined in the reference publication: Biochemistry, Daniel Voet/Judith G. Voet, p. 250, John Wyley & Sons, 1990. The oligomeric structure (G)ₓ may be in any isomeric form, whether it is optical isomerism, geometrical isomerism or positional isomerism; it may also represent a mixture of isomers.

In formula (II) as defined above, the group R₁—O— is linked to G via the anomeric carbon of the saccharide residue, so as to form an acetal function.

According to a particular aspect in the definition of formula (II) as defined above, G represents a reducing sugar residue chosen from glucose, dextrose, sucrose, fructose, idose, gulose, galactose, maltose, isomaltose, maltotriose, lactose, cellobiose, mannose, ribose, xylose, arabinose, lyxose, allose, altrose, dextran and tallose; and more particularly G represents a reducing sugar residue chosen from glucose, xylose and arabinose residues.

According to an even more particular aspect, in the definition of formula (II) representing composition (C₁) included in composition (E₁) that is the subject of the present invention, x represents a decimal number greater than or equal to 1.05 and less than or equal to 2.5, more particularly greater than or equal to 1.05 and less than or equal to 2.0 and even more particularly greater than or equal to 1.25 and less than or equal to 2.0.

According to an even more particular aspect, in the definition of formula (II) as defined above, R₁ represents a radical chosen from at least one of the elements of the group consisting of n-dodecyl, n-tetradecyl, n-hexadecyl, n-octadecyl, n-eicosyl, n-docosyl, 2-hexyldecyl, 2-octyldecyl, 2-hexyldodecyl, 2-octyldodecyl and 2-decyltetradecyl radicals; G represents a reducing sugar residue chosen from glucose and xylose residues and x represents a decimal number greater than or equal to 1.05 and less than or equal to 2.5.

According to an even more particular aspect, composition (E₁) as defined previously is characterized in that said emulsifying system (S) consists of an alkylpolyglycoside composition (C₁) represented by formula (II):

R₁—O-(G)ₓ-H (II)

in which x represents a decimal number between 1.05 and 2.5, G represents a xylose residue and R₁ represents a 2-octyldodecyl radical, said composition (C₁) consisting of a mixture of compounds represented by formulae (I₁), (I₂), (I₃), (I₄) and (I₅):

R₁—O-(G)₁-H (II₁)

R₁—O-(G)₂-H (II₂)

R₁—O-(G)₃-H (II₃)

R₁—O-(G)₄-H (II₄)

R₁—O-(G)₅-H (II₅)

in the respective molar proportions a₁, a₂, a₃, a₄ and a₅ such that:
the sum a₁+a₂+a₃+a₄+a₅ is equal to 1 and that
the sum a₁+2a₂+3a₃+4a₄+5a₅ is equal to x.

In the definition of composition (E₁) which is the subject of the present invention, the term “alkylpolyglycoside and fatty alcohol composition” means a composition (C₂) comprising, per 100% of its mass:
from 10% to 50% by mass, more particularly from 15% to 40% by mass and even more particularly from 20% to 30% by mass of at least one composition (C₁) represented by formula (II) as defined previously,
from 90% to 50% by mass, more particularly from 85% to 60% by mass and even more particularly from 80% to 70% by mass of at least one fatty alcohol of formula (III):

R′₁—OH (III),

in which R′₁, which may be identical to or different from R₁, represents a saturated or unsaturated, linear or branched hydrocarbon-based aliphatic radical comprising from 12 to 36 carbon atoms, optionally substituted with one or more hydroxyl groups.

According to a particular aspect, in the definition of formula (II) representing composition (C₁) included in composition (C₂), R₁ represents a radical chosen from n-dodecyl, n-tetradecyl, n-hexadecyl, n-octadecyl, n-eicosyl, n-docosyl, 2-hexyldecyl, 2-octyldecyl, 2-hexyldodecyl, 2-octyldodecyl and 2-decyltetradecyl radicals, G represents a reducing sugar residue chosen from glucose and xylose residues and x represents a decimal number greater than or equal to 1.05 and less than or equal to 2.5

According to a more particular aspect, in the definition of formula (II) representing composition (C₁) included in composition (C₂), R₁ represents a 2-octyldodecyl radical, G represents a xylose residue and x represents a decimal number greater than or equal to 1.05 and less than or equal to 2.5.

According to a more particular aspect, in the definition of the fatty alcohol of formula (III) as defined above, R′₁ represents a radical chosen from n-dodecyl, n-tetradecyl, n-hexadecyl, n-octadecyl, n-eicosyl, n-docosyl, 2-hexyldecyl, 2-octyldecyl, 2-hexyldodecyl, 2-octyldodecyl and 2-decyltetradecyl radicals, R′₁ most particularly represents a 2-octyldodecyl radical.

According to an even more particular aspect, a subject of the invention is a composition (E₁) as defined previously, characterized in that said emulsifying system (S) consists of a composition (C₂) comprising, per 100% of its mass:
from 10% to 50% by mass of at least one alkylpolyglycoside composition (C₁) represented by formula (II):

R₁—O-(G)ₓ-H (II)

in which x represents a decimal number between 1.05 and 2.5, G represents a xylose residue and R₁ represents a 2-octyldodecyl radical, said composition consisting of a mixture of compounds represented by formulae (I₁), (I₂), (I₃), (I₄) and (I₅):

R₁—O-(G)₁-H (II₁)

R₁—O-(G)₂-H (II₂)

R₁—O-(G)₃-H (II₃)

R₁—O-(G)₄-H (II₄)

R₁—O-(G)₅-H (II₅)

in the respective molar proportions a₁, a₂, a₃, a₄ and a₅ such that:
the sum a₁+a₂+a₃+a₄+a₅ is equal to 1 and that
the sum a₁+2a₂+3a₃+4a₄+5a₅ is equal to x; and
from 90% to 50% by mass of at least one fatty alcohol of formula (III):

R′₁—OH (III),

in which R′₁ represents a 2-octyldodecyl radical.

In the definition of composition (E₁) that is the subject of the present invention, the term “polyglycerol ester” means a compound of formula (IV):

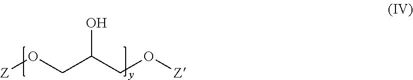

in which Z represents an acyl radical of formula R₂—C(═O)—, in which R₂ represents a linear or branched, saturated or unsaturated hydrocarbon-based aliphatic radical comprising from 11 to 35 carbon atoms and more particularly a radical chosen from dodecanoyl, tetradecanoyl, hexadecanoyl, octadecanoyl, eicosanoyl, docosanoyl, oleyl, linoleyl, linolenoyl and isostearyl radicals, Z′ represents an acyl radical of formula R₂—C(═O)— as defined above, with Z′ identical to or different from Z, or a hydrogen atom, and y represents an integer greater than or equal to 2 and less than or equal to 20.

According to a more particular aspect, the compound of formula (IV) is chosen from the elements of the group consisting of decaglyceryl oleate, decaglyceryl isostearate, decaglyceryl monolaurate, decaglyceryl monolinoleate and decaglyceryl monomyristate.

In the definition of composition (E₁) that is the subject of the present invention, the term “alkoxylated polyglycerol esters” means a compound of formula (V):

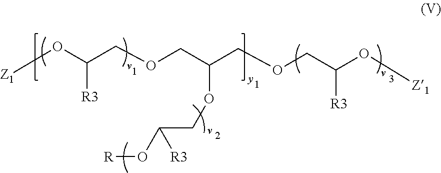

in which Z₁ represents an acyl radical of formula R′₂—C(═O)—, in which R′₂ represents a saturated or unsaturated, linear or branched hydrocarbon-based aliphatic radical comprising from 11 to 35 carbon atoms, and more particularly a radical chosen from dodecanoyl, tetradecanoyl, hexadecanoyl, octadecanoyl, eicosanoyl, docosanoyl, oleyl, linoleyl, linolenoyl and isostearyl radicals, Z₁′ represents an acyl radical of formula R′₂—C(═O)— as defined above, with Z₁′ identical to or different from Z₁, or a hydrogen atom, R₃ represents a hydrogen atom, a methyl radical or an ethyl radical, y₁ represents an integer greater than or equal to 2 and less than or equal to 20, v₁, v₂ and v₃, which may be identical or different, represent an integer greater than or equal to 0 and less than or equal to 50, and the sum [(y₁·v₁)+(y₁·v₂)+v₃)] is an integer greater than or equal to 1 and less than or equal to 50.

In the definition of composition (E₁) that is the subject of the present invention, the term “polyglycol polyhydroxystearates” denotes a compound of formula (VI):

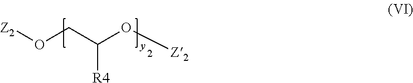

in which y₂ represents an integer greater than or equal to 2 and less than or equal to 50, R₄ represents a hydrogen atom, a methyl radical or an ethyl radical, Z₂ represents a radical of formula (VII):

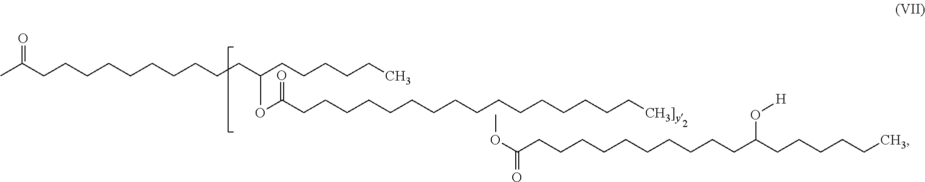

in which y′₂ represents an integer greater than or equal to 0 and less than or equal to 10, more particularly greater than or equal to 1 and less than or equal to 10 and Z′₂ represents a radical of formula (VII) as defined above, with Z₂′ identical to or different from Z₂, or a hydrogen atom.

In the definition of composition (E₁) that is the subject of the present invention, the term “polyglyceryl polyhydroxystearate” denotes a compound represented by formula (VIII):

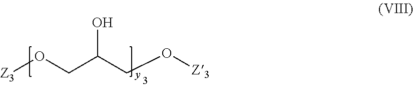

in which Z₃ represents a radical of formula (VII) as defined above, Z′₃ represents a radical of formula (VII) as defined above, with Z₃′ identical to or different from Z₃, or a hydrogen atom, y₃ represents an integer greater than or equal to 2 and less than or equal to 20.

In the definition of composition (E₁) that is the subject of the present invention, the term “alkoxylated polyglyceryl polyhydroxystearate” denotes a compound represented by formula (IX):

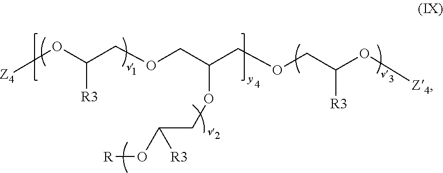

in which Z₄ represents a radical of formula (VII) as defined above, Z′₄ represents a radical of formula (VII) as defined above, with Z₄′ identical to or different from Z₄, or a hydrogen atom, y₄ represents an integer greater than or equal to 2 and less than or equal to 20, v′₁, v′₂ and v′₃, which may be identical or different, represent an integer greater than or equal to 0 and less than or equal to 50, and the sum [(y₄·v′₁)+(y₄·v′₂)+v′₃)] is an integer greater than or equal to 1 and less than or equal to 50.

In the definition of composition (E₁) that is the subject of the present invention, the term “polyethylene glycol-alkyl glycol copolymer” denotes a compound represented by formula (X):

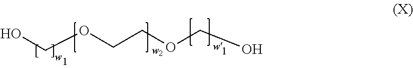

in which w₁ and w′₁, which may be identical or different, represent an integer greater than or equal to 1 and less than or equal to 50, more particularly greater than or equal to 1 and less than or equal to 25, w₂ represents an integer greater than or equal to 1 and less than or equal to 100, more particularly greater than or equal to 1 and less than or equal to 50.

According to another particular aspect, a subject of the invention is a composition (E₁) as defined previously, characterized in that said emulsifying system (S) consists of a composition (C₃) comprising, per 100% of its mass:

from 15% to 25% by mass of at least one composition (C₁) represented by formula (II):

R₁—O-(G)ₓ-H (II)

in which x represents a decimal number between 1.05 and 2.5, G represents a xylose residue and R₁ represents a 2-octyldodecyl radical, said composition (C₁) consisting of a mixture of compounds represented by formulae (I₁), (I₂), (I₃), (I₄) and (I₅):

R₁—O-(G)₁-H (II₁)

R₁—O-(G)₂-H (II₂)

R₁—O-(G)₃-H (II₃)

R₁—O-(G)₄-H (II₄)

R₁—O-(G)₅-H (II₅)

in the respective molar proportions a₁, a₂, a₃, a₄ and a₅, such that:
the sum a₁+a₂+a₃+a₄+a₅ is equal to 1 and that
the sum a₁+2a₂+3a₃+4a₄+5a₅ is equal to x;
from 55% to 65% by mass of at least one fatty alcohol of formula (III):

R′₁—OH (III),

in which R′₁ represents a 2-octyldodecyl radical;

from 10% to 30% by mass of at least one polyglycol polyhydroxystearate represented by formula (VI):

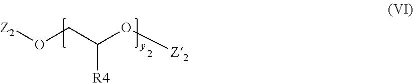

in which y₂ represents an integer greater than or equal to 2 and less than or equal to 50, R₄ represents a hydrogen atom, a methyl radical or an ethyl radical, Z₂ represents a radical of formula (VII):

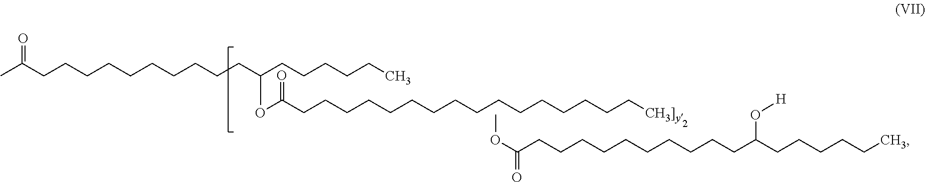

in which y′₂ represents an integer greater than or equal to 0 and less than or equal to 10, more particularly greater than or equal to 1 and less than or equal to 10, Z′₂ represents a radical of formula (VII) as defined above, with Z₂′ identical to or different from Z₂, or a hydrogen atom.

According to another particular aspect, a subject of the invention is a composition (E₁) as defined previously, characterized in that the dynamic viscosity of said composition (E₁), measured at a temperature of 20° C. using a Brookfield LVT viscometer at a speed of 6 rpm, is greater than or equal to 500 mPa·s and less than or equal to 40 000 mPa·s.

A subject of the invention is also the use of composition (E₁) as defined previously, for cleaning, protecting and/or caring for the skin, the hair, the scalp or mucous membranes.

Composition (E₁) that is the subject of the present invention as defined previously is intended for topical use, and may be incorporated in any type of cosmetic, dermocosmetic, dermopharmaceutical or pharmaceutical formulation intended for topical use, or alternatively in any type of support intended to be placed in contact with the skin (paper, wipe, textile, transdermal device, etc.).

The term “for topical use” means that composition (E₁) is used by application to the skin, the hair, the scalp or mucous membranes, whether this involves direct application in the case of a cosmetic, dermocosmetic, dermopharmaceutical or pharmaceutical formulation or indirect application, for example in the case of a body hygiene product, a skincare product or a skin-protecting product, which is in the form of a textile article, for example a wipe, or in paper form, for example a paper for sanitary use.

Composition (E₁) that is the subject of the present invention may be packaged in pressurized form in an aerosol device or in a device of “pump-bottle” type, in a device equipped with a perforated wall, for example a grille, or in a device equipped with a ball applicator (known as a “roll-on”).

Composition (E₁) that is the subject of the present invention may be used as cleansing or makeup-removing milks, as cleansing or makeup-removing lotions, as foaming gels for the face or the body, as shampoo for cleaning the hair and/or the scalp, as hair conditioner for treating the hair and/or the scalp, as bubble bath, as cream, milk or lotion for caring for or protecting the face, the hands and the body, for example as an antisun protecting agent, as a self-tanning agent, as an antiaging agent, as an antiwrinkle agent, as a calmative or as a moisturizer.

Composition (E₁) that is the subject of the present invention may also comprise excipients and/or active principles usually used in the field of formulations for topical use, in particular cosmetic, dermocosmetic, pharmaceutical or dermopharmaceutical formulations.

This is why, according to another particular mode, a subject of the invention is a composition (E₁) as defined previously, characterized in that it also comprises one or more auxiliary compounds chosen from foaming and/or detergent surfactants, thickening and/or gelling surfactants, thickening and/or gelling agents, stabilizers, film-forming compounds, solvents and cosolvents, hydrotropic agents, plasticizers, emulsifiers and coemulsifiers, opacifiers, nacreous agents, overfatting agents, sequestrants, chelating agents, antioxidants, fragrances, essential oils, preserving agents, conditioning agents, deodorants, bleaching agents for decolorizing bodily hair and the skin, active principles for providing a treating and/or protective action on the skin or the hair, sunscreens, mineral fillers or pigments, particles that give a visual effect or that are intended for encapsulating active agents, exfoliant particles, texture agents, optical brighteners and insect repellents.

As examples of foaming and/or detergent surfactants optionally present in composition (E₁) that is the subject of the present invention, mention may be made of topically acceptable anionic, cationic, amphoteric or nonionic foaming and/or detergent surfactants usually used in this field of activity.

Among the foaming and/or detergent anionic surfactants that may be combined with composition (E₁) that is the subject of the present invention, mention may be made of alkali metal salts, alkaline-earth metal salts, ammonium salts, amine salts, amino alcohol salts of alkyl ether sulfates, of alkyl sulfates, of alkylamido ether sulfates, of alkylarylpolyether sulfates, of monoglyceride sulfates, of α-olefin sulfonates, of paraffin sulfonates, of alkyl phosphates, of alkyl ether phosphates, of alkyl sulfonates, of alkylamide sulfonates, of alkylaryl sulfonates, of alkyl carboxylates, of alkylsulfosuccinates, of alkyl ether sulfosuccinates, of alkylamide sulfosuccinates, of alkyl sulfoacetates, of alkyl sarcosinates, of acylisethionates, of N-acyl taurates, of acyl lactylates, of N-acylamino acid derivatives, of N-acyl peptide derivatives, of N-acyl protein derivatives or of fatty acids.

Among the foaming and/or detergent amphoteric surfactants optionally present in composition (E₁) that is the subject of the present invention, mention may be made of alkylbetaines, alkylamidobetaines, sultaines, alkylamidoalkylsulfobetaines, imidazoline derivatives, phosphobetaines, amphopolyacetates and amphopropionates.

Among the foaming and/or detergent cationic surfactants optionally present in composition (E₁) that is the subject of the present invention, mention may be made particularly of quaternary ammonium derivatives.

Among the foaming and/or detergent nonionic surfactants that may be present in composition (E₁) that is the subject of the present invention, mention may be made more particularly of alkylpolyglycosides comprising a linear or branched, saturated or unsaturated aliphatic radical and comprising from 8 to 12 carbon atoms; castor oil derivatives, polysorbates, copra amides and N-alkylamines.

As examples of thickening and/or gelling surfactants optionally present in composition (E₁) that is the subject of the present invention, mention may be made of:
optionally alkoxylated alkylpolyglycoside fatty esters, and most particularly ethoxylated methylpolyglucoside esters such as PEG 120 methyl glucose trioleate and PEG 120 methyl glucose dioleate sold, respectively, under the names Glucamate™ LT and Glumate™ DOE120;
alkoxylated fatty esters such as PEG 150 pentaerythrityl tetrastearate sold under the name Crothix™ DS53, or PEG 55 propylene glycol oleate sold under the name Antil™ 141;
fatty-chain polyalkylene glycol carbamates such as PPG 14 laureth isophoryl dicarbamate sold under the name Elfacos™ T211, PPG 14 palmeth 60 hexyl dicarbamate sold under the name Elfacos™ GT2125.

As examples of emulsifying surfactants that may be present in composition (E₁) that is the subject of the present invention, mention may be made of nonionic surfactants, anionic surfactants and cationic surfactants.

As examples of emulsifying nonionic surfactants that may be present in composition (E₁) that is the subject of the present invention, mention may be made of fatty acid esters of sorbitol, for example the products sold under the names Montane™80 and Montane™85 and Montane™60; ethoxylated castor oil and ethoxylated hydrogenated castor oil, for example the product sold under the name Simulsol™ 989; compositions comprising glyceryl stearate and stearic acid poly(ethoxylated) with between 5 mol and 150 mol of ethylene oxide, for example the composition comprising stearic acid (ethoxylated) with 135 mol of ethylene oxide and glyceryl stearate sold under the name Simulsol™ 165; ethoxylated sorbitan esters, for example the products sold under the name Montanox™, mannitan esters; ethoxylated mannitan esters; sucrose esters; methylglucoside esters.

As examples of emulsifying anionic surfactants that may be present in composition (E₁) that is the subject of the present invention, mention may be made of decyl phosphate, cetyl phosphate sold under the name Amphisol™, glyceryl stearate citrate; cetearyl sulfate; the arachidyl/behenyl phosphates and arachidyl/behenyl alcohols composition sold under the name Sensanov™ WR; soaps, for example sodium stearate or triethanolammonium stearate, salified N-acylamino acid derivatives, for instance stearoyl glutamate.

As examples of emulsifying cationic surfactants that may be present in composition (E₁) that is the subject of the present invention, mention may be made of amine oxides, Quaternium-82 and the surfactants described in patent application WO 96/00719 and mainly those in which the fatty chain comprises at least 16 carbon atoms.

As examples of opacifying and/or nacreous agents that may be present in composition (E₁) that is the subject of the present invention, mention may be made of sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate and fatty alcohols comprising from 12 to 22 carbon atoms.

As examples of texture agents that may be present in composition (E₁) that is the subject of the present invention, mention may be made of N-acylamino acid derivatives, for example lauroyl lysine sold under the name Aminohope™ LL, octenyl starch succinate sold under the name Dryflo™, myristyl polyglucoside sold under the name Montanov 14, cellulose fibers, cotton fibers, chitosan fibers, talc, sericite and mica.

As examples of solvents and cosolvents that may be present in composition (E₁) that is the subject of the present invention, mention may be made of water, organic solvents, for example glycerol, diglycerol, glycerol oligomers, ethylene glycol, propylene glycol, butylene glycol, hexylene glycol, diethylene glycol, xylitol, erythritol, sorbitol, water-soluble alcohols such as ethanol, isopropanol or butanol, and mixtures of water and of said organic solvents.

As examples of active principles that may be present in composition (E₁) that is the subject of the present invention, there are:
vitamins and derivatives thereof, for example retinol (vitamin A) and esters thereof (for example retinyl palmitate), ascorbic acid (vitamin C) and esters thereof, sugar derivatives of ascorbic acid (for example ascorbyl glucoside), tocopherol (vitamin E) and esters thereof (for example tocopheryl acetate), vitamins B3 or B10 (niacinamide and derivatives thereof);
compounds with lightening or depigmenting action on the skin, for example Sepiwhite™ MSH, arbutin, kojic acid, hydroquinone, Vegewhite™, Gatuline™, Synerlight™, Biowhite™, Phytolight™, Dermalight™, Clariskin™, Melaslow™, Dermawhite™, Ethioline, Melarest™, Gigawhite™, Albatine™ and Lumiskin™,
compounds with a calmative action, for instance Sepicalm™ S, allantoin and bisabolol;
antiinflammatory agents;
compounds with moisturizing action, for example urea, hydroxy ureas, glycerol, polyglycerols, glycerol glucoside, diglycerol glucoside or polyglyceryl glucosides;
compounds with slimming or lipolytic action, for instance caffeine or derivatives thereof, Adiposlim™ or Adipoless™,
N-acyl proteins; N-acyl peptides, for example Matrixil™; N-acylamino acids; partial hydrolyzates of N-acyl proteins; amino acids; peptides; total protein hydrolyzates;
plant extracts rich in tannins, in polyphenols and/or in isoflavones, for example grape extracts, pine extracts, wine extracts, olive extracts; soybean extracts, for example Raffermine™; wheat extracts, for example Tensine™ or Gliadine™; terpene-rich plant extracts; freshwater or seawater algal extracts; marine extracts in general such as coral;
essential waxes; bacterial extracts; ceramides or phospholipids;
compounds with antimicrobial action or purifying action, for example Lipacide™ CBG, Lipacide™ UG, Sepicontrol™ A5; Octopirox™ or Sensiva™ SC50;
compounds with an energizing or stimulating property, for instance Physiogényl™, panthenol and derivatives thereof, for instance Sepicap™ MP;
antiaging active agents, for instance Sepilift™ DPHP, Lipacide™ PVB, Sepivinol™, Sepivital™, Manoliva™, Phyto-Age™, Timecode™, Survicode™,
anti-photoaging active agents; active agents for protecting the integrity of the dermo-epidermal junction;
active agents for increasing the synthesis of components of the extracellular matrix, for example collagen, elastins and glycosaminoglycans;
active agents acting favorably on chemical cell communication, for instance cytokines, or physical cell communication, for instance integrins;
active agents that create a “heating” sensation on the skin, for instance skin capillary circulation activators (for example nicotinic acid derivatives) or products that create a sensation of “freshness” on the skin (for example menthol and derivatives thereof);
active agents that improve the skin capillary circulation, for example venotonic agents; draining active agents; active agents for decongestive purposes, for example Ginkgo biloba, ivy, common horse chestnut, bamboo, ruscus, butcher's-broom, Centella asiatica, fucus, rosemary or willow extracts;
skin tanning or browning agents, for example dihydroxyacetone, isatin, alloxan, ninhydrin, glyceraldehyde, mesotartaric aldehyde, glutaraldehyde or erythrulose.

As examples of thickeners and/or gelling agents that may be present in composition (E₁) that is the subject of the present invention, mention may be made of homopolymers or copolymers of acrylic acid or of acrylic acid derivatives, homopolymers or copolymers of acrylamide, homopolymers or copolymers of acrylamidomethylpropanesulfonic acid, of vinyl monomer, of trimethylaminoethyl acrylate chloride, hydrocolloids of plant or biosynthetic origin, for example xanthan gum, karaya gum, carrageenates or alginates; galactomannans, for example tara gum, guar gum, fenugreek gum, locust bean gum or cassia gum; silicates; cellulose and derivatives thereof; starch and hydrophilic derivatives thereof; polyurethanes.

As examples of deodorants that may be present in composition (E₁) that is the subject of the present invention, mention may be made of alkali metal silicates, zinc salts such as zinc sulfate, zinc gluconate, zinc chloride or zinc lactate; quaternary ammonium salts such as cetyltrimethylammonium salts or cetylpyridinium salts; glycerol derivatives such as glyceryl caprate, glyceryl caprylate and polyglyceryl caprate; 1,2-decanediol, 1,3-propanediol; salicylic acid; sodium bicarbonate; cyclodextrins; metallic zeolites; Triclosan™; aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum sulfate, sodium aluminum lactate, complexes of aluminum chlorohydrate and of glycol, for instance the complex of aluminum chlorohydrate and of propylene glycol, the complex of aluminum dichlorohydrate and of propylene glycol, the complex of aluminum sesquichlorohydrate and of propylene glycol, the complex of aluminum chlorohydrate and of polyethylene glycol, the complex of aluminum dichlorohydrate and of polyethylene glycol, or the complex of aluminum sesquichlorohydrate and of polyethylene glycol.

As examples of sunscreens that may be present in composition (E₁) that is the subject of the present invention, mention may be made of all those featured in the cosmetic directive 76/768/EEC modified by annex VII.

Among the organic sunscreens that may be present in composition (E₁) that is the subject of the present invention, mention may be made of:
benzoic acid derivatives, for instance para-aminobenzoic acids (PABA), especially monoglyceryl esters of PABA, ethyl esters of N,N-propoxy PABA, ethyl esters of N,N-diethoxy PABA, ethyl esters of N,N-dimethyl PABA, methyl esters of N,N-dimethyl PABA and butyl esters of N,N-dimethyl PABA;
anthranilic derivatives, for instance homomenthyl-N-acetyl anthranilate;
salicylic acid derivatives, for instance amyl salicylate, homomenthyl salicylate, ethylhexyl salicylate, phenyl salicylate, benzyl salicylate and p-isopropanolphenyl salicylate;
cinnamic acid derivatives, for instance ethylhexyl cinnamate, ethyl-4-isopropyl cinnamate, methyl-2,5-diisopropyl cinnamate, p-methoxypropyl cinnamate, p-methoxyisopropyl cinnamate, p-methoxyisoamyl cinnamate, p-methoxyoctyl cinnamate (p-methoxy 2-ethylhexyl cinnamate), p-methoxy-2-ethoxyethyl cinnamate, p-methoxycyclohexyl cinnamate, ethyl-α-cyano-β-phenyl cinnamate, 2-ethylhexyl-α-cyano-β-phenyl cinnamate or glyceryl di-para-methoxy-mono-2-ethylhexanoyl cinnamate;
benzophenone derivatives, for instance 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenylbenzophenone-2-carboxylate, 2-hydroxy-4-n-octyloxybenzophenone, 4-hydroxy-3-carboxybenzophenone; 3-(4′-methylbenzylidene)-d,l-camphor, 3-(benzylidene)-d,l-camphor, camphor benzalkonium methosulfate; urocanic acid, ethyl urocanate; the family of sulfonic acid derivatives, for instance 2-phenylbenzimidazole-5-sulfonic acid and salts thereof;
triazine derivatives, for instance hydroxyphenyltriazine, (ethylhexyloxyhydroxyphenyl)(4-methoxyphenyl)triazine, 2,4,6-trianillino(p-carbo-2′-ethylhexyl-1′-oxy)-1,3,5-triazine, 4,4-((6-(((1,1-dimethylethyl)amino)carbonyl)phenyl)amino)-1,3,5-triazine-2,4-diyldiimino)bis-(2-ethylhexyl)benzoate, 2-phenyl-5-methylbenzoxazole, 2,2′-hydroxy-5-methylphenylbenzotriazole, 2-(2′-hydroxy-5′-t-octylphenyl)benzotriazole, 2-(2′-hydroxy-5′-methyphenyl)benzotriazole; dibenzazine; dianisoylmethane, 4-methoxy-4″-t-butylbenzoylmethane; 5-(3,3-dimethyl-2-norbornylidene)-3-pentan-2-one;
diphenylacrylate derivatives, for instance 2-ethylhexyl-2-cyano-3,3-diphenyl-2-propenoate, ethyl-2-cyano-3,3-diphenyl-2-propenoate;
polysiloxanes, for instance benzylidene siloxane malonate.

Among the inorganic sunscreens, also known as “mineral sunblocks”, that may be present in composition (E₁) that is the subject of the present invention, mention may be made of titanium oxides, zinc oxides, cerium oxide, zirconium oxide, yellow, red or black iron oxides and chromium oxides. These mineral sunblocks may be micronized or non-micronized, may or may not have undergone surface treatments and may be optionally in the form of aqueous or oily predispersions.

A subject of the invention is also the use of composition (E₁) as defined previously for the cosmetic treatment of the skin, the hair and/or mucous membranes and more particularly for cleansing, protecting and/or caring for the skin, the hair, the scalp or mucous membranes.

A subject of the invention is also a process for preparing composition (E₁) as defined previously, comprising the following steps:

a step a) of preparing the fatty phase (A₂) by mixing all of the elements constituting it in the desired proportions. This mixing step is generally performed at a temperature of greater than or equal to 20° C. and less than or equal to 80° C., more particularly greater than or equal to 20° C. and less than or equal to 60° C., and even more particularly greater than or equal to 20° C. and less than or equal to 40° C.; it is performed with mechanical stirring at a moderate speed of greater than or equal to 50 rpm and less than or equal to 100 rpm;

a step b) of preparing the aqueous phase (A₁) of all of the elements constituting it in the desired proportions. This mixing step is generally performed at a temperature of greater than or equal to 20° C. and less than or equal to 80° C., more particularly greater than or equal to 20° C. and less than or equal to 60° C., and even more particularly greater than or equal to 20° C. and less than or equal to 40° C.; it is performed with mechanical stirring at a moderate speed of greater than or equal to 500 rpm and less than or equal to 3000 rpm. In particular, the aqueous phase (A₁) obtained after step b) has a dynamic viscosity, measured at 20° C. with a Brookfield LV viscometer at a speed of 6 rpm, of greater than or equal to 200 mPa·s and less than or equal to 40 000 mPa·s, more particularly greater than or equal to 1000 mPa·s and less than or equal to 40 000 mPa·s, and even more particularly greater than or equal to 2000 mPa·s and less than or equal to 40 000 mPa·s;

a step c) during which the fatty phase (A₂) is added to the aqueous phase (A₁) at a temperature of greater than or equal to 20° C. and less than or equal to 80° C., more particularly greater than or equal to 20° C. and less than or equal to 60° C., and even more particularly greater than or equal to 20° C. and less than or equal to 40° C., with mechanical stirring at a moderate speed of greater than or equal to 50 rpm and less than or equal to 400 rpm, so as to obtain composition (E₁).

### I)—Preparation of Crosslinked Anionic Polvelectrolvtes

I-1 Terpolymer of ammonium 2-methyl-2-[(1-oxo-2-propenyl)amino]-1-propanesulfonate, of N,N-dimethylacrylamide and of Tetraethoxylated Lauryl Methacrylate [AMPSNH₄/DMAM/LMA(4EO) 77.4/19.2/3.4 mol], Crosslinked with Trimethylolpropane Triacrylate (TMPTA)

592 g of an aqueous solution containing 15% by mass of ammonium 2-methyl-2-[(1-oxo-2-propenyl)amino]-1-propanesulfonate (AMPSNH₄) in a mixture of tert-butanol/water (97.5/2.5 by volume), 10.1 g of N,N-dimethylacrylamide (DMAM), 4.2 g of tetraethoxylated lauryl methacrylate [LMA(4EO)] and 0.75 g of TMPTA are placed in a reactor maintained at 25° C. with stirring. After a time sufficient to achieve good homogenization of the solution, it is deoxygenated by sparging with nitrogen heated to 70° C. 0.42 g of dilauroyl peroxide is then added and the reaction medium is then maintained for about 60 minutes at 70° C. and then for 2 hours at 80° C. After cooling, the powder formed during the polymerization is filtered off and dried to obtain the desired product, referred to hereinbelow as: “Polyelectrolyte (PA₁)”.

I-2 Terpolymer of ammonium 2-methyl-2-[(1-oxo-2-propenyl)amino]-1-propanesulfonate and of N,N-dimethylacrylamide [AMPSNH₄/DMA 80/20 mol] Crosslinked with Trimethylolpropane Triacrylate (TMPTA)

592 g of an aqueous solution containing 15% by mass of AMPSNH₄ in a tert-butanol/water mixture (97.5/2.5 by volume), 10.1 g of DMAM and 0.75 g of TMPTA are placed in a reactor maintained at 25° C. with stirring. After a time sufficient to achieve good homogenization of the solution, it is deoxygenated by sparging with nitrogen heated to 70° C. 0.42 g of dilauroyl peroxide is then added and the reaction medium is then maintained for about 60 minutes at 70° C. and then for 2 hours at 80° C. After cooling, the powder formed during the polymerization is filtered off and dried to obtain the desired product, referred to hereinbelow as: “Polyelectrolyte (PA₂)”.

### II)—Preparation and Evaluation of Water-in-Oil Emulsions According to the Invention and of Comparative Water-in-Oil Emulsions

### II-1 Preparation of Water-in-Oil Emulsions

Four water-in-oil emulsions according to the invention, noted (F₁) to (F₄), and six water-in-oil emulsions according to the prior art, noted (F′₁) to (F′₆), are prepared, the mass proportions of the constituents of which are given in table 1 below, the mass contents of the polyelectrolytes being indicated as percentages of polymer solids, using the following process:

The constituents of the fatty phase are successively placed in a beaker and mixed at a temperature of 20° C., using a mechanical stirrer equipped with a stirring rotor of impeller type, at a speed of 100 rpm. The glycerol and the water are mixed at room temperature in a beaker using a mechanical stirrer at a speed of 2000 rpm and the thickener is then gradually added. Stirring is continued for a time making it possible to produce an aqueous phase in the form of a homogeneous gel. The fatty phase is added in a single portion to the aqueous gel, at room temperature and at a moderate stirring speed (75 to 300 rpm) with a stirrer equipped with a rotor of anchor type. This stirring is then maintained for ten minutes and no cooling step is required. Emulsions (F′₃) and (F′₄) thus prepared are representative of the prior art teaching of the European patent application published under the number EP 1 459 801 A2.

| | (F₁) | (F₂) | (F₃) | (F₄) | (F′₁) |
|---|---|---|---|---|---|
| Fatty phase | | | | | |
| Lanol ™ 99⁽¹⁾ | 5.3% | 5.3% | 5.3% | 5.3% | 5.3% |
| Easynov ™⁽²⁾ | 2% | 2% | 2% | 2% | 2% |
| Euxyl ™ PE9010⁽³⁾ | 1% | 1% | 1% | 1% | 1% |
| Fragrance | 0.1% | 0.1% | 0.1% | 0.1% | 0.1% |
| Aqueous phase | | | | | |
| Glycerol | 14% | 14% | 14% | 14% | 14% |
| Polyelectrolyte | 1.5% | 1% | 0.7% | 0.5% | 2.1% |
| (PA₁) | | | | | |
| Water | qs 100% | qs 100% | qs 100% | qs 100% | qs 100% |
| NaOH | qs | qs | qs | qs | qs |
| | pH = 5.5 | pH = 5.5 | pH = 5.5 | pH = 5.5 | pH = 5.5 |
| | (F′₂) | (F′₃) | (F′₄) | (F′₅) | (F′₆) |
| Fatty p | | | | | |
| Lanol ™ 99 | 5.3% | 5.3% | 5.3% | 5.3% | 5.3% |
| Easynov ™ | 2% | 2% | 2% | 2% | 2% |
| Euxyl ™ PE9010 | 1% | 1% | 1% | 1% | 1% |
| Fragrance | 0.1% | 0.1% | 0.1% | 0.1% | 0.1% |
| Aqueous phase | | | | | |
| Glycerol | 14% | 14% | 14% | 14% | 14% |
| Polyelectrolyte (PA₂) | 0.7% | 0% | 0% | 2% | 0.% |
| Simulgel ™EG⁽⁴⁾ | 0% | 0.7% | 0.3% | 0% | 0.% |
| Pemulen TR2⁽⁵⁾ | 0% | 0% | 0% | 0% | 2% |
| Water | qs 100% | qs 100% | qs 100% | qs 100% | qs 100% |
| NaOH | qs | qs | qs | qs | qs |
| | pH = 5.5 | pH = 5.5 | pH = 5.5 | pH = 5.5 | pH = 5.5 |

⁽¹⁾Lanol ™ 99 (INCI name: isononyl isononanoate): ester used as oily phase in the cosmetic composition preparation and distributed by the company SEPPIC;
⁽²⁾Easynov ™ (INCI name: octyldodecanol, octyldodecyl xyloside and PEG-30 dipolyhydroxystearate): emulsifying composition sold by the company SEPPIC, comprising, per 100% of its mass, from 55% to 65% by mass of 2-octyldodecanol, 15% to 25% by mass of 2-octyldodecyl polyxyloside, 10% to 30% by mass of PEG-30 dipolyhydroxystearate;
⁽³⁾Euxyl ™ PE9010 (INCI name: phenoxyethanol & ethylhexyl glycerol): composition used as preserving agent;
⁽⁴⁾Simulgel ™ EG (INCI name: sodium acrylate/sodium acryloyldimethyltaurate copolymer and isohexadecane and polysorbate 80): reverse latex approximately comprising, per 100% of its mass, about 38% by mass of a copolymer of partially salified acrylic acid and of the sodium salt of 2-methyl-2-[(1-oxo-2-propenyl)amino]-1-propanesulfonic acid, crosslinked with methylenebisacrylamide;
⁽⁵⁾Pemulen ™TR2 (INCI name: acrylates/C10-30 alkyl acrylate crosspolymer) is a high molecular weight polymeric emulsifier, crosslinked copolymer of acrylic acid and of a hydrophobic comonomer.

### II-2 Demonstration of the Properties of the Water-in-Oil Emulsions (F₁) to (F₄) According to the Invention and of the Water-in-Oil Emulsions (F′₁) to (F′₆) According to the Prior Art

### II-2.1 Characterization of the Appearance and Viscosity of the Water-in-Oil Emulsions (F₁) to (F₄) According to the Invention and of the Water-in-Oil Emulsions (F′₁) to (F′₆) According to the Prior Art

The emulsions (F₁) to (F₄) and (F′₁) to (F′₆) obtained according to the process described previously were then stored in an insulated air-conditioned chamber regulated at a temperature of 20° C. for 7 days. After this period of 7 days, the appearance (APP) of each prepared emulsion is observed and the dynamic viscosity (μ) of each emulsion is measured (in mPa·s). The emulsions are then replaced and stored in the same insulated air-conditioned chamber regulated at a temperature of 20° C. up to 3 months. After a period of one month, each emulsion is removed from the air-conditioned chamber to observe its appearance and to measure its dynamic viscosity at 20° C., using a viscometer at 20° C. (Brookfield LVT, speed 6).

### II-2.2 Characterization of the Sense of Emulsions (F₁) to (F₄) According to the Invention and of Emulsions (F′₁) to (F′₆) According to the Prior Art

The conductivity (a) of emulsions (F₁) to (F₄) according to the invention and of emulsions (F′₁) to (F′₆) is measured at 20° C., after a period of storage of said emulsions of one day in an insulated air-conditioned chamber regulated at a temperature of 20° C., using an LF 196™ brand conductimeter from the company WTW equipped with a Tétracon™ 96 electrode.

If, for a given emulsion, (a) is less than or equal to 0.5 μS·cm⁻¹, it is considered that the emulsion is not conductive and consequently that the outer phase is not the aqueous phase but the oily phase; such an emulsion for which (a) is less than or equal to 0.5 μS·cm⁻¹ is thus a water-in-oil emulsion.

### II-2.3 Evaluation of the Consistency of a Composition

The term “consistency (γ)” of a composition means the resistance to flow of said composition, which may be characterized by empirical observations, such as visual observation of the flow of the composition and/or by measuring a flow threshold using a rheometer of cone-plate type connected to data processing software.

II-2.3.1 Evaluation of the Consistency of the Composition by Visual Observation

a) Principle of the Method

The consistency may be evaluated by visual observation of the flow of the evaluated composition which is contained in a flask following 180° rotation of said flask.

b) Experimental Protocol

50 grams of the test composition are placed in a 100 mL transparent glass flask, which is closed with a screw stopper. The duly trained and experienced experimenter rotates by 180° the flask containing the test composition over a period of 3 seconds and notes his observation. He then rotates once again by 180° the flask containing the test composition.

c) Expressing the Results
If, during the 3 seconds, during which the containing flask is rotated by 180°, the test composition flows spontaneously, or if, during the 3 seconds, during which the containing flask is rotated by 180°, the test composition does not flow spontaneously but flows after three manual impulses given on the bottom of the flask by the experimenter, the working composition is termed as being “liquid”;
if, during the 3 seconds, during which the containing flask is rotated by 180°, the test composition does not flow spontaneously and if it does not flow after three manual impulses given on the bottom of the flask by the experimenter, the test composition is termed as being “compact”.

II-2.3.2 Evaluation of the Consistency of a Composition by Measuring a Flow Threshold

a) Principle of the Method

The consistency may be evaluated by measuring the flow threshold (FT), performed using an AR2000 model rheometer sold by the company TA Instruments, and equipped with a Peltier plate for controlling the temperature and a cone connected to data processing software.

b) Experimental Protocol

The test composition is deposited on the plate 24 hours after its manufacture.

The AR2000 model rheometer consists of a cone-plate system with an air stage, the plate equipped with a Peltier system to control the measuring temperature. The cone is made of aluminum, measures 6 centimeters in diameter and its angle is 1°. The sample positioned in the gap of the plate and the cone is sheared in “oscillation” mode and more particularly in stress sweep mode, at a temperature of 25° C., at an oscillation frequency of 1 Hz. The force sensor measures continuously the resistance phase shift of the product with respect to the stress imposed in the gap. Exploitation of these measurements by the software makes it possible to deduce the viscoelasticity of the sample, namely the value G′ (in Pa) of the elastic solid component and the value G″ (in Pa) of the viscous liquid component. When the value of G″ is greater than the value of G′, the sample behaves like a liquid, and flows. During a sweep under increasing stress, from 0.1 to 100 Pa, the sample behaves like a solid at low stresses, and then like a liquid at higher stresses, beyond a threshold known as the flow threshold. The value of the threshold stress, or flow threshold (in Pa), is extrapolated from the curve of G′, as the point of crossing of the tangents at the point of inflection. The accuracy is of the order of 1 Pa.

c) Expressing the Results
If the flow threshold value, measured under the experimental conditions as described above, is greater than or equal to a value of 40 Pa, the test composition is considered as being “compact”;
if (FT)<40 Pa, the test composition is considered as being “liquid”.

### II-2.4 Results Obtained for the Water-in-Oil Emulsions (F₁) to (F₄) According to the Invention and for the Water-in-Oil Emulsions (F′₁) to (F′₆) According to the Prior Art

The evaluation methods described in paragraphs II-2.1 and II-2.2 were applied to the water-in-oil emulsions (F₁) to (F₄) according to the invention and to the water-in-oil emulsions (F′₁) to (F′₆) according to the prior art.

The results obtained are given in table 2 below.

TABLE 2

| | (F₁) | (F₂) | (F₃) | (F₄) | (F′₁) |
|---|---|---|---|---|---|
| (γ) at 1 day | Liquid | Liquid | Liquid | Liquid | Liquid |
| (visual) | | | | | |
| (σ) at 1 day | 0.2 | 0.2 | 0.1 | 0.1 | 0.3 |
| (FT) at 1 day | <40 Pa | <40 Pa | <40 Pa | <40 Pa | <40 Pa |
| (APP) at 7 | Homogeneous | Homogeneous | Homogeneous | Homogeneous | Homogeneous |
| days | | | | | |
| (μ) at 7 days | 24700 | 28600 | 34100 | 29800 | 20600 |
| (APP) at 1 | Homogeneous | Homogeneous | Homogeneous | Homogeneous | Homogeneous |
| month | | | | | |
| (μ) at 1 month | 22400 | 26300 | 33300 | 28300 | 11400 |
| (APP) at 3 | Homogeneous | Homogeneous | Homogeneous | Homogeneous | Homogeneous |
| months | | | | | |
| (μ) at 3 months | 18920 | 22100 | 31200 | 24200 | 10200 |
| | (F′₂) | (F′₃) | (F′₄) | (F′₅) | (F′₆) |
| (γ) at 1 day | Liquid | Compact | Liquid | Liquid | Liquid |
| (visual) | | | | | |
| (σ) at 1 day | 0.3 | 0.1 | 0.3 | 0.2 | 1.8 |
| (FT) at 1 day | <40 Pa | >40 Pa | <40 Pa | <40 Pa | <40 Pa |
| (APP) at 7 | Homogeneous | Homogeneous | Homogeneous | Homogeneous | Phase shift |
| days | | | | | |
| (μ) at 7 days | 19600 | 60300 | 52100 | 10700 | N.M. |
| (APP) at 1 | Homogeneous | Homogeneous | Homogeneous | Homogeneous | Phase shift |
| month | | | | | |
| (μ) at 1 month | 12800 | 59800 | 49200 | 8790 | N.M. |
| (APP) at 3 | Homogeneous | Homogeneous | Homogeneous | Phase shift | Phase shift |
| months | | | | | |
| (μ) at 3 months | n.m. | 62200 | n.m | N.M. | N.M. |

n.m.: not measured;
N.M: notmeasurable

Furthermore, it was observed that when the water-in-oil formulations (F₁), (F₂), (F₃) and (F₄) according to the invention are applied to the skin, they are characterized by the formation of an oily film on said skin, which persists throughout the spreading time.

### II-2.5 Analysis of the Results

The water-in-oil emulsions (F₁), (F₂), (F₃) and (F₄) according to the invention are thus characterized:
by stability of their water-in-oil form after 3 months of storage at a temperature of 20° C.; the appearance observed after this storage period is still homogeneous;
by a consistency judged as being “fluid” after one day at 20° C. according to the visual observation method described in paragraph II-2.3.1 of the present description;
by a flow threshold value, measured under the experimental conditions as described in paragraph II-2.3.2 of the experimental section of the present description, strictly less than a value of 40 Pa,
by dynamic viscosity values measured after 7 days of storage at 20° C., using a Brookfield LV viscometer at 20° C. and at a speed of 6 rpm, of between 24 700 mPa·s (for (F₁)) and 34 100 mPa·s (for (F₃));
by limited reductions in dynamic viscosity after storage for three months at 20° C., since the decreases in the value of said dynamic viscosities measured after 3 months of storage at 20° C. (Brookfield LV, 6 rpm at 20° C.) are not 25% greater than the dynamic viscosity value measured after 7 days of storage under the same conditions. Thus, for storage at 20° C., the decrease in the dynamic viscosity value between the 7^{th} day of storage and the end of the 3^{rd} month of storage is 15.5% for (F₁), 22.7% for (F₂), 8.5% for (F₃) and 18.8% for (F₄).

The emulsion (F′₁) differs from the emulsions according to the invention by an amount of crosslinked anionic polyelectrolyte “Polyelectrolyte (PA₁)” greater than 2% per 100% of the mass of the emulsion, i.e. greater than 1.65% per 100% of the mass of the aqueous phase, which leads unexpectedly to a 50.5% reduction in the dynamic viscosity of said emulsion (F′₁) between the 7^{th} day and the end of the 3^{rd} month of storage at 20° C.

When the water-in-oil emulsions are prepared according to the same protocol as described above, differing from the water-in-oil emulsions according to the invention only in the nature of the thickener, in this instance the anionic polyelectrolyte (PA₂) differing from the anionic polyelectrolyte (PA₁) by the absence of associative monomer in the polymer backbone, the corresponding emulsion (F′₂) shows a 34% reduction in the dynamic viscosity of said emulsion (F′₂) between the 7th day and the end of the 1^{st} month of storage at 20° C. When the amount of anionic polyelectrolyte (PA₂) is increased to 2%, as opposed to 0.7% for the anionic polyelectrolyte (PA₁), the water-in-oil emulsion obtained (F′₅) undergoes phase separation after 3 months of storage at 20° C.

When water-in-oil emulsions are prepared according to the same process as described above, differing from the water-in-oil emulsions according to the invention only in the nature of the thickener, in this instance a crosslinked anionic polyelectrolyte based on partially salified acrylic acid and 2-methyl-2-[(1-oxo-2-propenyl)amino]-1-propanesulfonic acid partially salified in ammonium form, said water-in-oil emulsions do not have a liquid consistency ((F′₃) judged as being compact) or else are characterized by an excessively high level of viscosity (F′₄).

When an emulsion (F′₆) is prepared according to the same process as described above, differing from the water-in-oil emulsions according to the invention only in the nature of the thickener, in this instance a polyelectrolyte having the INCI name acrylates/C10-30 alkyl acrylate crosspolymer, which is a crosslinked copolymer of acrylic acid and of a hydrophobic comonomer, the emulsion (F′₆) obtained is not a water-in-oil emulsion and it is unstable from the first day of storage.

### III) Applicative Examples

In the following formulations, the percentages are expressed by weight of the formulation.

III-1 Antisun water-in-oil emulsion

| | |
|---|---|
| Diisopropyl adipate: | 12% |
| Ethylhexyl salicylate: | 5% |
| Ethylhexyl methoxycinnamate: | 5% |
| Ethylhexyl 4-(N,N-dimethylamino)benzoate: | 8% |
| Butylmethoxydibenzoylmethane: | 2% |
| PEG30 dipolyhydroxystearate: | 0.4% |
| Fluidanov ™ 20X: | 1.2% |
| Polyelectrolyte (PA₁): | 0.7% |
| Glycerol: | 1.5% |
| Euxyl ™ PE9010: | 1% |
| Water: | qs 100% |

III-2 Cushion-texture bodycare water-in-oil emulsion

| | |
|---|---|
| Isononyl isononanoate: | 4% |
| Fragrance: | 0.6% |
| (Ethylhexyl)glycerol: | 0.2% |
| Easynov ™: | 2.5% |
| Polyelectrolyte (PA₁): | 0.6% |
| Glycerol: | 3% |
| Dye: | 0.07% |
| 95° ethanol: | 15% |
| Water: | qs 100% |

III-3 Deep winter body milk water-in-oil emulsion

| | |
|---|---|
| Isononyl isononanoate: | 5.3% |
| Fragrance: | 0.1% |
| Phenoxyethanol & (ethylhexyl)glycerol: | 1% |
| Easynov ™: | 2% |
| Polyelectrolyte (PA₁): | 1.5% |
| Glycerol: | 14% |
| Water: | qs 100% |

III-4 Moisturizing care water-in-oil emulsion

| | |
|---|---|
| Isononyl isononanoate: | 6% |
| Fragrance: | 0.1% |
| Phenoxyethanol & (ethylhexyl)glycerol: | 1% |
| Easynov ™: | 1.5% |
| Polyelectrolyte (PA₁): | 0.5% |
| Aquaxyl ™: | 3% |
| Water: | qs 100% |

III-5 Antiperspirant water-in-oil emulsion

| | |
|---|---|
| Isononyl isononanoate: | 6% |
| (Ethylhexyl)glycerol: | 1% |
| Easynov ™: | 2.5% |
| Polyelectrolyte (PA₁): | 0.5% |
| Aquaxyl ™: | 3% |
| Water: | qs 100% |
| Aluminum chlorohydrate (50%) | 30% |

III-6 Fragrancing water-in-oil emulsion

| | |
|---|---|
| Isononyl isononanoate: | 6% |
| (Ethylhexyl)glycerol: | 1% |
| Easynov ™: | 2.5% |
| Polyelectrolyte (PA₁): | 0.5% |
| Aquaxyl ™: | 3% |
| Water: | qs 100% |
| Ethanol: | 40% |
| Fragrance: | 2.0% |

III-7 Lightening water-in-oil emulsion

| | |
|---|---|
| Isononyl isononanoate: | 6% |
| Fragrance: | 0.1% |
| Phenoxyethanol & (ethylhexyl)glycerol: | 1% |
| Easynov ™: | 1.5% |
| Polyelectrolyte (PA₁): | 0.5% |
| Sepiwhite ™MSH: | 2% |
| Water: | qs 100% |

III-8 Deodorant water-in-oil emulsion

| | |
|---|---|
| Isononyl isononanoate: | 6% |
| Fragrance: | 0.1% |
| Phenoxyethanol & (ethylhexyl)glycerol: | 1% |
| Easynov ™: | 1.5% |
| Polyelectrolyte (PA₁): | 0.5% |
| Lipacide ™UG: | 1% |
| Water: | qs 100% |

Aquaxyl™ (INCI name: xylitylglucoside & anhydroxylitol & xylitol) is a moisturizer sold by the company SEPPIC.
Sepiwhite™ MSH (INCI name: ω-undecylenoyl phenylalanine) is a skin-lightening agent sold by the company SEPPIC.
Lipacide™ UG (INCI name: undecylenoyl glycine) is a deodorant and dermo-purifying agent sold by the company SEPPIC.

1. A composition (E₁) which is in the form of a water-in-oil emulsion, characterized in that it comprises per 100% of its mass:
from 60% to 98% by mass of an aqueous phase (A₁) comprising, per 100% of its mass, from 0.05% to 1.65% by mass of a crosslinked anionic polyelectrolyte (P) derived from the polymerization, in the presence of at least one crosslinking agent, of 2-methyl-2-[(1-oxo-2-propenyl)amino]-1-propanesulfonic acid which is partially or totally salified, with at least one neutral monomer chosen from N,N-dialkylacrylamides, in which each of the alkyl groups comprises between one and four carbon atoms, and at least one monomer of formula (I): 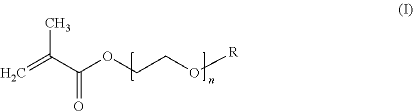
in which R represents a linear or branched alkyl radical comprising from eight to twenty carbon atoms and n represents an integer greater than or equal to one and less than or equal to twenty;
from 2% to 40% by mass of a fatty phase (A₂) comprising, per 100% of its mass, from 1.25% to 50% by mass of an emulsifying system (S) comprising one or more emulsifying surfactants selected from alkylpolyglycoside compositions, alkylpolyglycoside and fatty alcohol compositions, polyglycerol esters, alkoxylated polyglycerol esters, polyglycol polyhydroxystearates, polyglycerol polyhydroxystearates, alkoxylated polyglycerol polyhydroxystearates, polyethylene glycol-alkyl glycol copolymers; from 50% to 98.75% by mass of at least one oil and optionally of at least one wax.

2. The composition (E₁) as defined in claim 1, characterized in that it comprises, per 100% of its mass:
from 60% to 98% by mass of an aqueous phase (A₁) comprising, per 100% of its mass, from 0.05% to 1.65% by mass of a crosslinked anionic polyelectrolyte (P) derived from the polymerization, in the presence of a crosslinking agent, of 2-methyl-2-[(1-oxo-2-propenyl)amino]-1-propanesulfonic acid, which is partially or totally salified, with a neutral monomer chosen from N,N-dialkylacrylamides, in which each of the alkyl groups comprises between one and four carbon atoms, and a monomer of formula (I);
from 2% to 40% by mass of a fatty phase (A₂) comprising, per 100% of its mass, from 1.25% to 50% by mass of an emulsifying system (S) comprising one or more emulsifying surfactants selected from alkylpolyglycoside compositions, alkylpolyglycoside and fatty alcohol compositions, polyglycerol esters, alkoxylated polyglycerol esters, polyglycol polyhydroxystearates, polyglycerol polyhydroxystearates, alkoxylated polyglycerol polyhydroxystearates, polyethylene glycol-alkyl glycol copolymers; from 50% to 98.75% by mass of an oil and optionally of a wax.

3. The composition (E₁) as defined in claim 1, characterized in that said crosslinked anionic polyelectrolyte (P) comprises, per 100 mol % of its constituent monomers:
from 20 mol % to 80 mol % of monomer units derived from 2-methyl-2-[(1-oxo-2-propenyl)amino]-1-propanesulfonic acid, which is partially or totally salified;
from 15 mol % to 75 mol % of monomer units derived from at least one neutral monomer chosen from N,N-dialkylacrylamides, in which each of the alkyl groups comprises between one and four carbon atoms;
from 0.5 mol % to 5 mol % of monomer units derived from at least one monomer of formula (I).

4. The composition (E₁) as defined in claim 1, characterized in that said neutral monomer is N,N-dimethylacrylamide.

5. The composition (E₁) as defined in claim 1, characterized in that said monomer of formula (I) is tetraethoxylated lauryl methacrylate.

6. The composition (E₁) as defined in claim 1, characterized in that said crosslinked anionic polyelectrolyte (P) is terpolymer of 2-methyl-2-[(1-oxo-2-propenyl)amino]-1-propanesulfonic acid partially salified in ammonium salt form, of N,N-dimethylacrylamide and of tetraethoxylated lauryl methacrylate, crosslinked with trimethylolpropanetriacrylate.

7. The composition (E₁) as defined in claim 1, characterized in that said crosslinked anionic polyelectrolyte (P) comprises, per 100 mol %:
from 60 mol % to 80 mol % of monomer units derived from 2-methyl-2-[(1-oxo-2-propenyl)amino]-1-propanesulfonic acid partially salified in ammonium form,
from 15 mol % to 39.5 mol % of monomer units derived from N,N-dimethylacrylamide, and
from 0.5 mol % to 5 mol % of monomer units derived from tetraethoxylated lauryl methacrylate.

8. The composition (E₁) as defined in claim 1, characterized in that said emulsifying system (S) consists of an alkylpolyglycoside composition (C₁) represented by formula (II):
R₁—O-(G)ₓ-H (II)
in which x represents a decimal number between 1.05 and 2.5, G represents a xylose residue and R₁ represents a 2-octyldodecyl radical, said composition (C₁) consisting of a mixture of compounds represented by formulae (I₁), (I₂), (I₃), (I₄) and (I₅):
R₁—O-(G)₁-H (II₁)
R₁—O-(G)₂-H (II₂)
R₁—O-(G)₂-H (II₂)
R₁—O-(G)₄-H (II₄)
R₁—O-(G)₅-H (II₅)
in the respective molar proportions a₁, a₂, a₃, a₄ and a₅ such that:
the sum a₁+a₂+a₃+a₄+a₅ is equal to 1 and that
the sum a₁+2a₂+3a₃+4a₄+5a₅ is equal to x.

9. The composition (E₁) as defined in claim 1, characterized in that said emulsifying system (S) consists of a composition (C₂) comprising, per 100% of its mass:
from 10% to 50% by mass of at least one alkylpolyglycoside composition (C₁) represented by formula (II):
R₁—O-(G)ₓ-H (II)
in which x represents a decimal number between 1.05 and 2.5, G represents a xylose residue and R₁ represents a 2-octyldodecyl radical, said composition consisting of a mixture of compounds represented by formulae (I₁), (I₂), (I₃), (I₄) and (I₅):
R₁—O-(G)₁-H (II₁)
R₁—O-(G)₂-H (II₂)
R₁—O-(G)₃-H (II₃)
R₁—O-(G)₄-H (II₄)
R₁—O-(G)₅-H (II₅)
in the respective molar proportions a₁, a₂, a₃, a₄ and a₅ such that:
the sum a₁+a₂+a₃+a₄+a₅ is equal to 1 and that
the sum a₁+2a₂+3a₃+4a₄+5a₅ is equal to x; and
from 90% to 50% by mass of at least one fatty alcohol of formula (III):
R′₁—OH (III),
in which R′₁ represents a 2-octyldodecyl radical.

10. The composition (E₁) as defined in claim 1, characterized in that said emulsifying system (S) consists of a composition (C₃) comprising, per 100% of its mass:
from 15% to 25% by mass of at least one composition (C₁) represented by formula (II):
R₁—O-(G)ₓ-H (II)
in which x represents a decimal number between 1.05 and 2.5, G represents a xylose residue and R₁ represents a 2-octyldodecyl radical, said composition (C₁) consisting of a mixture of compounds represented by formulae (I₁), (I₂), (I₃), (I₄) and (I₅):
R₁—O-(G)₁-H (II₁)
R₁—O-(G)₂-H (II₂)
R₁—O-(G)₃-H (II₃)
R₁—O-(G)₄-H (II₄)
R₁—O-(G)₅-H (II₅)
in the respective molar proportions a₁, a₂, a₃, a₄ and a₅ such that:
the sum a₁+a₂+a₃+a₄+a₅ is equal to 1 and that
the sum a₁+2a₂+3a₃+4a₄+5a₅ is equal to x;
from 55% to 65% by mass of at least one fatty alcohol of formula (III):
R′₁—OH (III),
in which R′₁ represents a 2-octyldodecyl radical;
from 10% to 30% by mass of at least one polyglycolpolyhydroxystearate represented by formula (VI): 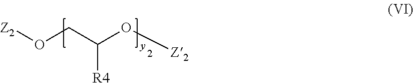
in which y₂ represents an integer greater than or equal to 2 and less than or equal to 50, R₄ represents a hydrogen atom, a methyl radical or an ethyl radical, Z₂ represents a radical of formula (VII): 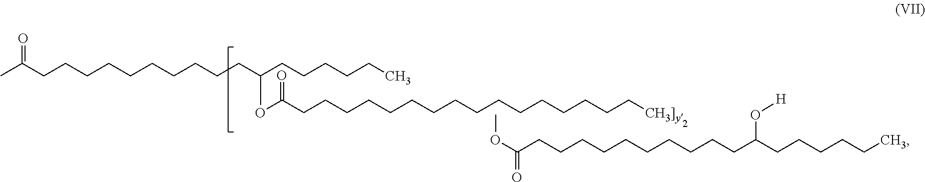
in which y′₂ represents an integer greater than or equal to 0 and less than or equal to 10, more particularly greater than or equal to 1 and less than or equal to 10, Z′₂ represents a radical of formula (VII) as defined above, with Z₂′ identical to or different from Z₂, or a hydrogen atom.

11. The composition (E₁) as defined in claim 1, characterized in that it also comprises one or more auxiliary compounds chosen from foaming and/or detergent surfactants, thickening and/or gelling surfactants, thickening and/or gelling agents, stabilizers, film-forming compounds, solvents and cosolvents, hydrotropic agents, plasticizers, emulsifiers and coemulsifiers, opacifiers, nacreous agents, overfatting agents, sequestrants, chelating agents, antioxidants, fragrances, essential oils, preserving agents, conditioning agents, deodorants, bleaching agents for decolorizing bodily hair and the skin, active principles for providing a treating and/or protective action on the skin or the hair, sunscreens, mineral fillers or pigments, particles that give a visual effect or that are intended for encapsulating active agents, exfoliant particles, texture agents, optical brighteners and insect repellents.

12. The composition (E₁) as defined in claim 1, characterized in that its dynamic viscosity, measured at a temperature of 20° C. using a Brookfield LVT viscometer at a speed of 6 rpm, is greater than or equal to 500 mPa·s and less than or equal to 40 000 mPa·s.

13. The use f composition (E₁) as defined in claim 1, further comprising an active agent for the cosmetic treatment of the skin, the hair and/or mucous membranes.

14. A method for cleansing, protecting and/or caring for the skin, the hair, the scalp or mucous membranes, comprising applying to the skin, the hair, the scalp or mucous membranes of a subject in need thereof, the composition (E₁) of claim 13.
